(19) United States
(12) Patent Application Publication
(10) Pub. No.: US 2023/0365484 A1
(43) Pub. Date: Nov. 16, 2023

(54) PROCESS OF PREPARING FLUOROETHER COMPOUNDS WITH UNSATURATED END GROUPS

(71) Applicant: MERCK PATENT GMBH, DARMSTADT (DE)

(72) Inventors: Reiner FRIEDRICH, Darmstadt (DE); Fabian KOCH, Darmstadt (DE); Laura SCHIMURA, Darmstadt (DE)

(73) Assignee: MERCK PATENT GMBH, DARMSTADT (DE)

(21) Appl. No.: 18/024,617

(22) PCT Filed: Sep. 6, 2021
(86) PCT No.: PCT/EP2021/074444

§ 371 Date: Mar. 3, 2023

(30) Foreign Application Priority Data
Sep. 9, 2020 (EP) 20195200.9

(51) Int. Cl. C07C 41/16; C07C 41/06; C07C 319/08

(52) U.S. Cl. CPC C07C 41/16; C07C 41/06; C07C 319/08

(57) ABSTRACT

The present invention relates to a process of preparing polyfluoroether compounds with unsaturated end groups and to compounds prepared by said process.

The present invention relates to a process of preparing polyfluoroether compounds with unsaturated end groups and to compounds prepared by said process.

Dirt-repellent coatings are a vital part for many technical applications. Often fluorinated material are used, because they have the unique characteristics of providing oil and water repellency at the same time. Out-door equipment, technical protection clothing like fireman suits, bullet proofed jackets or scrubs are only a few examples for these applications.

For dirt repellent coatings polyfluoroether acrylate or methacrylate monomers are often used. These monomers are usually prepared in a process comprising multiple reaction steps, including the reaction of a polyfluorinated ether with an acrylic or methacrylic acid derivative

However, with increasing number of process steps the yield of the final product is decreasing. This leads to an increase of the production costs, especially when costly starting materials have to be used. Due to their high safety and energy costs fluorinated compounds are generally among the most expensive products in organic chemistry.

It is therefore desirable to find an easy and cost-effective synthesis method for the preparation of polyfluoroether acrylates or methacrylates, or derivatives thereof with other unsaturated end groups, which allows to reduce the number of process steps, preferably to only one synthesis step, and to add the fluorinated ether in the final (or only) step.

This was achieved by a process as disclosed and claimed hereinafter.

The present invention thus relates to a process for preparing a compound of formula I

(Rf(CHF—CF₂Y)ₘ)ₙL(X)ₖ I

by reacting a compound of formula II

Rf(CHF═CF₂)ₘ II

with a compound of formula III

(HY)ₙL(X)ₖ III

in the presence of a base,

wherein the individual radicals, independently of each other and on each occurrence identically or differently, have the following meanings
Rf is a perfluorinated alkyl or alkylene group, preferably a perfluorinated C1-C30 alkyl or alkylene group, very preferably a perfluorinated C1-C15 alkyl or alkylene group, that optionally contains one or more hetero atoms which are preferably 0 atoms, Y is O or S, preferably 0,
L is a single bond or an at least divalent organic group, and
X is an ethylenically unsaturated group,
k is 1, 2 or 3, preferably 1 or 2, very preferably 1
m is 1 or 2, preferably 1,
n is 1, 2 or 3, preferably 1 or 2, very preferably 1,

The present invention further relates to novel compounds of formula I, II and III and their subformulae as described above and below, in particular compounds of formula I and III wherein X is an ethynyl (i.e. —C≡CH) group.

In the compounds of formula I and II wherein m is 1, Rf is preferably selected from the following groups:
CF₃—(CF₂)₀₋₃—, CF₃—(CF₂)₀₋₃—O—,
CF₃—(CF₂)₀₋₃—O—(CF₂)₁₋₃—,
CF₃—(CF₂)₀₋₃—O—(CF₂)₁₋₃—O—,
CF₃—(CF₂)₀₋₃—O—(CF₂)₁₋₃—O—CF₂—,
CF₃—(CF₂)₀₋₃—O—(CF₂—O)₁₋₈— and
CF₃—(CF₂)₀₋₃—O—(CF₂—O)₁₋₈—CF₂—.

In the compounds of formula I and II wherein m is 1, Rf is very preferably selected from the following groups:
CF₃—(CF₂)₁₋₂—, CF₃—(CF₂)₁₋₂—O—,
CF₃—O—(CF₂)₁₋₃—,
CF₃—O—(CF₂)₁₋₂—O—,
CF₃—(CF₂)₁₋₂—O—CF₂—,
CF₃—O—(CF₂)₁₋₂—O—CF₂—,
CF₃—O—(CF₂—O)₁₋₈— and
CF₃—O—(CF₂—O)₁₋₈—CF₂—.

In the compounds of formula I and II wherein m is 2, Rf is preferably selected from the following groups:
—(CF₂)₁₋₄—, —O—(CF₂)₁₋₃—O—,
—(CF₂)₁₋₃—(CF₂)₀₋₃—O—(CF₂)₁₋₃—,
O—(CF₂)₁₋₃—(CF₂)₀₋₃—O—(CF₂)₁₋₃—O—,
—(CF₂)₁₋₃—O—(CF₂)₁₋₃—O—CF₂—,
—CF₂—(CF₂)₀₋₃—O—(CF₂—O)₁₋₈— and
CF₂—(CF₂)₀₋₃—O—(CF₂—O)₁₋₈—CF₂—. (OK?)

In the compounds of formula I and II wherein m is 2, Rf is very preferably selected from the following groups:
—(CF₂)₁₋₃—, —(CF₂)₁₋₃—O—,
—O—(CF₂)₁₋₃—O—,
—CF₂—O—(CF₂)₁₋₂—O—,
—CF₂—(CF₂)₁₋₂—O—CF₂—,
—CF₂—O—(CF₂)₁₋₂—O—CF₂—,
—CF₂—O—(CF₂—O)₁₋₈— and
—CF₂—O—(CF₂—O)₁₋₈—CF₂—.

In the compounds of formula I and III, X is preferably an acrylate or methacrylate group, further preferably an ethynyl (i.e. —C≡CH) group.

In the compounds of formula I and III, the valence of the group L is given by the sum of n+k and is thus 2, 3, 4, 5 or 6. Preferably L is a divalent, trivalent or tetravalent group.

Preferably L is selected from a single bond or a saturated, straight-chain or branched, optionally hetero atoms, especially O and/or S atoms, and/or functional groups containing, organic group, especially preferably an alkylene group.

Especially preferably L is a saturated C1-C20, more preferably C1-C12, most preferably C1-C6, alkylene group that is straight-chain or branched, optionally contains one or more heteroatoms, preferably one or more O atoms, and optionally contains one or more functional groups.

Suitable and preferred functional groups include, without limitation, OH, epoxy, —Si(OMe)₃, —Si(OEt)₃, CO₂H or tertiary amino groups.

Very preferably L is selected from the group consisting of methylene, ethylene, propylene, isopropylene, n-butylene, isobutylene, sec-butylene, t-butylene, or pentylene or hexylene which are straight-chain or branched.

Particular preference is given to compounds of formula I, II and III wherein
m is 1 and Rf is selected from CF₃—(CF₂)₁₋₂—, CF₃—(CF₂)₁₋₂—O—, CF₃—O—(CF₂)₁₋₃—, CF₃—O—(CF₂)₁₋₃—O—, CF₃—(CF₂)₁₋₂—O—CF₂—, CF₃—O—(CF₂)₁₋₂—O—CF₂—, CF₃—O—(CF₂—O)₁₋₈— and CF₃—O—(CF₂—O)₁₋₈—CF₂—,
or m is 2 and Rf is selected from —(CF₂)₁₋₄—, —O—(CF₂)₁₋₃—O—, —(CF₂)₁₋₃—(CF₂)₀₋₃—O—(CF₂)₁₋₃—, —O—(CF₂)₁₋₃—(CF₂)₀₋₃—O—(CF₂)₁₋₃—O—, —(CF₂)₁₋₃—O—(CF₂)₁₋₃—O—CF₂—, —CF₂—(CF₂)₀₋₃—O—(CF₂—O)₁₋₈— and CF₂—(CF₂)₀₋₃—O—(CF₂—O)₁₋₈—CF₂—.
and
L is a C1-C6 alkylene group that is straight-chain or branched and optionally contains one or more hetero atoms, preferably one or more O atoms, and optionally contains a functional group.

Especially preferred compounds of formula I are selected from the following formulae

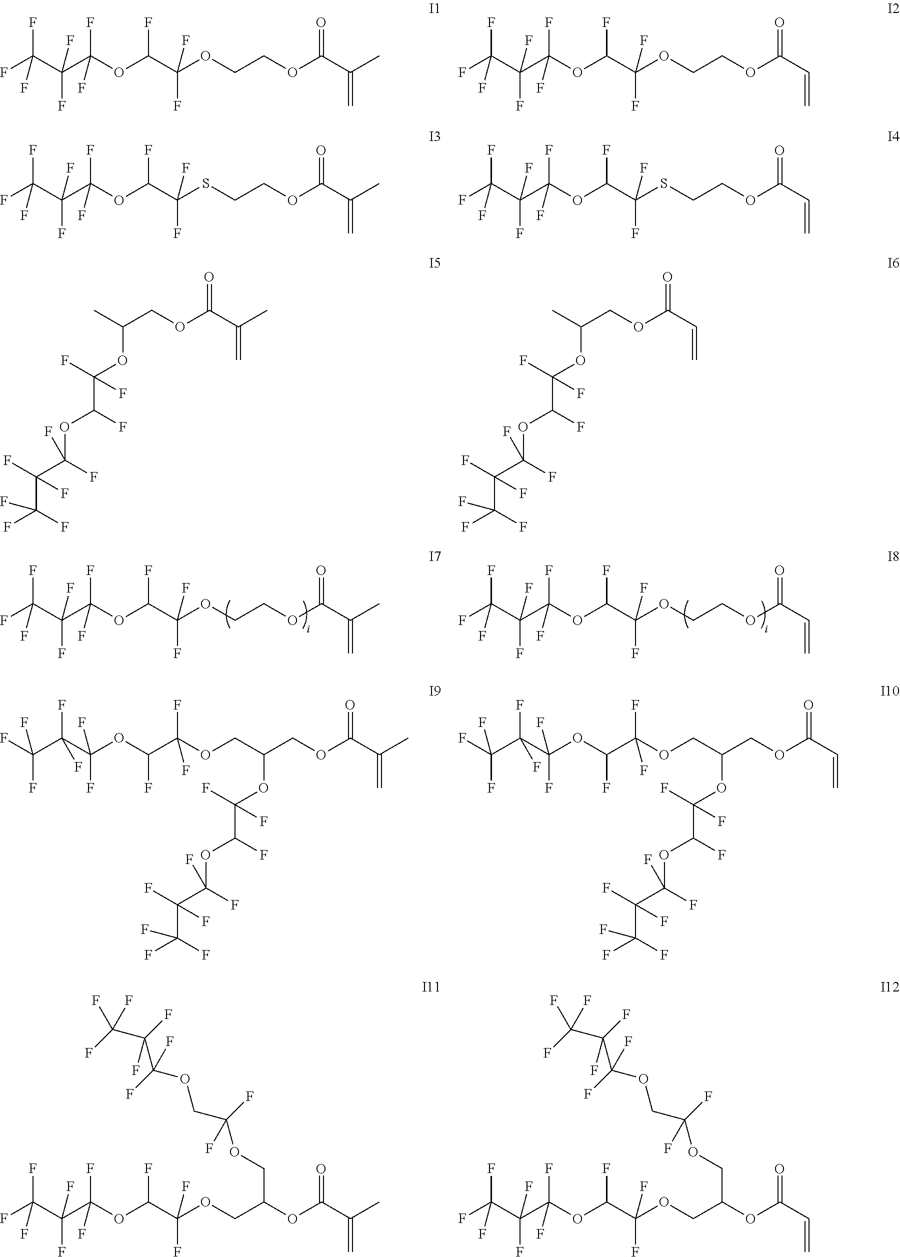

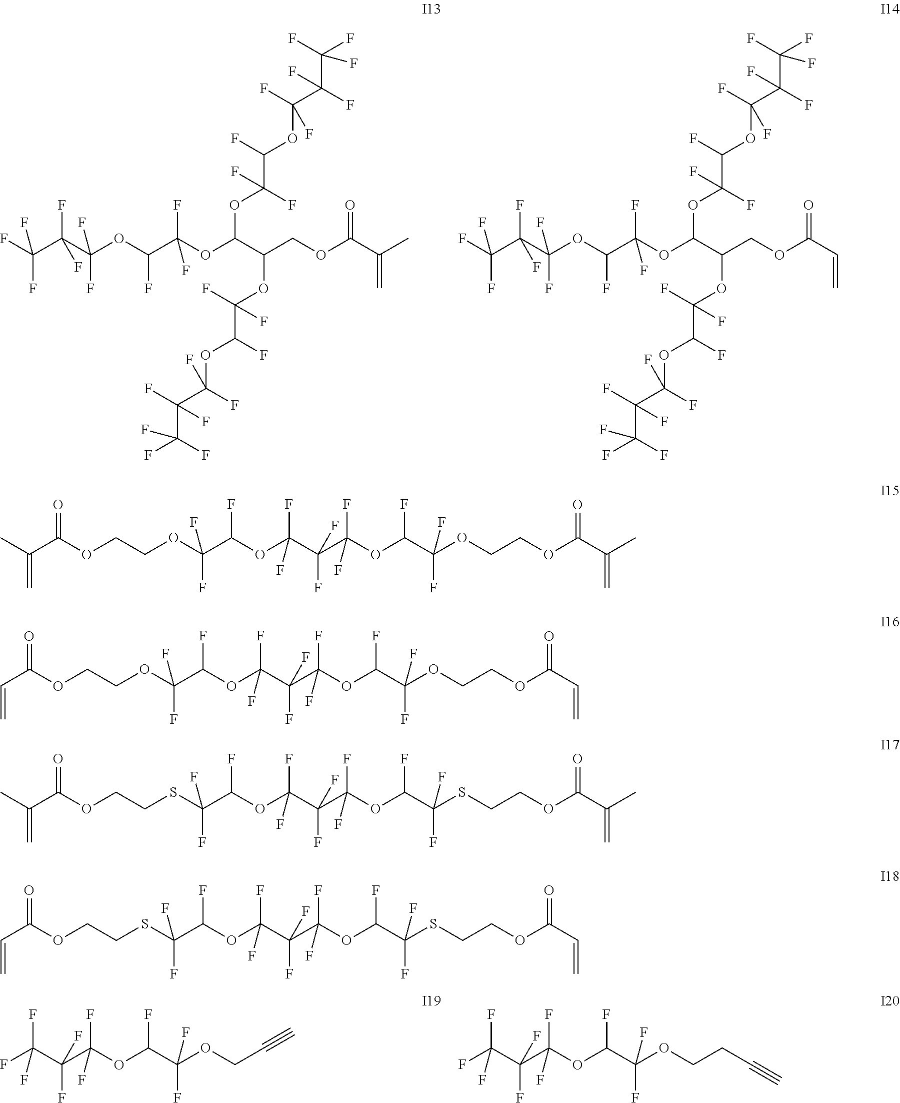

wherein i is 2-10.

Especially preferred compounds of formula II are selected from the following formulae.

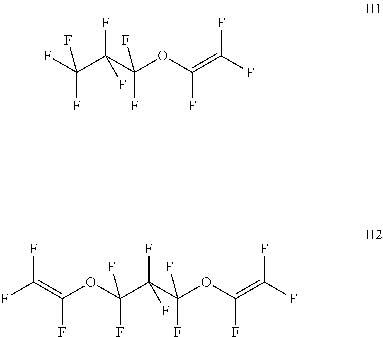 [SMILES: FC(F)=C(F)OC(F)(F)C(F)(F)C(F)(F)F ; FC(F)=C(F)OC(F)(F)C(F)(F)C(F)(F)OC(F)=C(F)F]

Especially preferred compounds of formula III are selected from the following formulae.

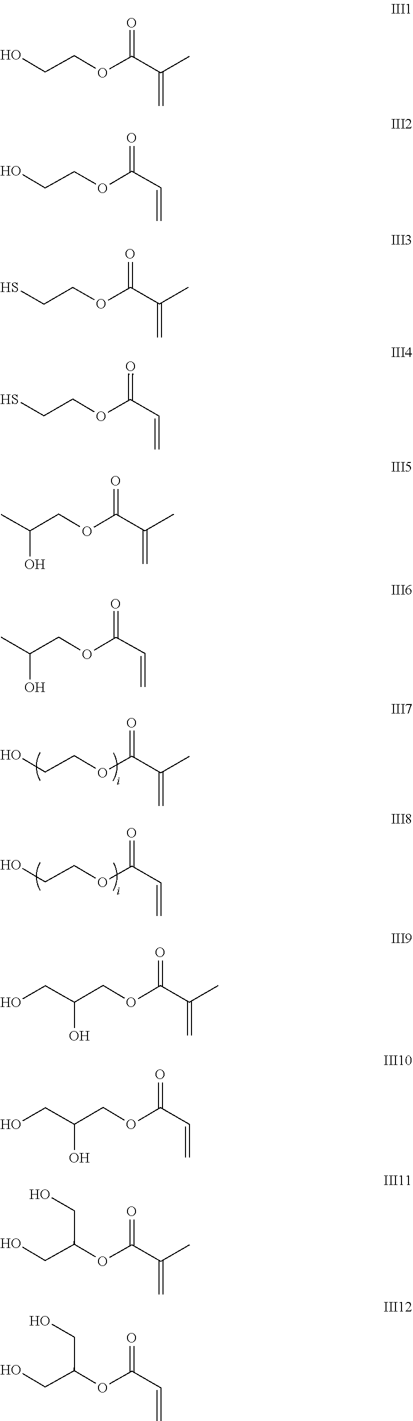

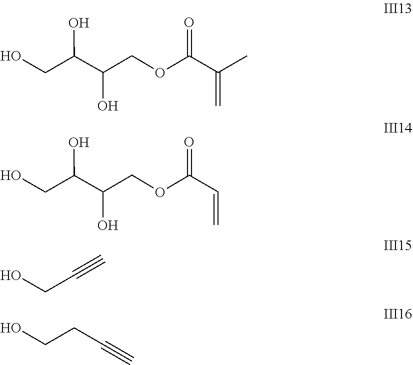

wherein i is 2-10.

The process according to the present invention is advantageous as it requires only one step to prepare the final product, as exemplarily illustrated in reaction Scheme 1 below for the compound of formula I1.

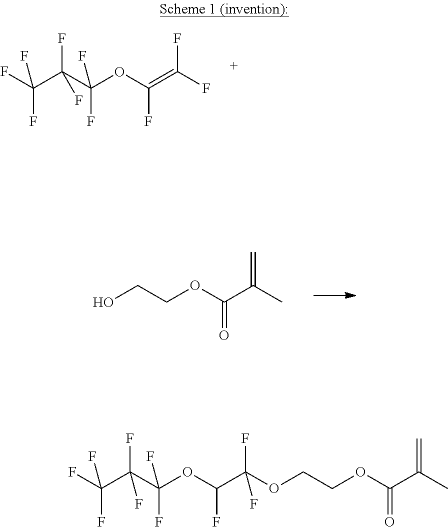

In contrast thereto, the methods hitherto known require two or even three steps, wherein the fluorinated educt is already employed in the first step, as exemplarily illustrated in reaction Schemes 2 and 3 below. This leads to a decrease in the yield of the final product relative to the amount of the employed fluorinated starting material and thus to an increase of the production costs.

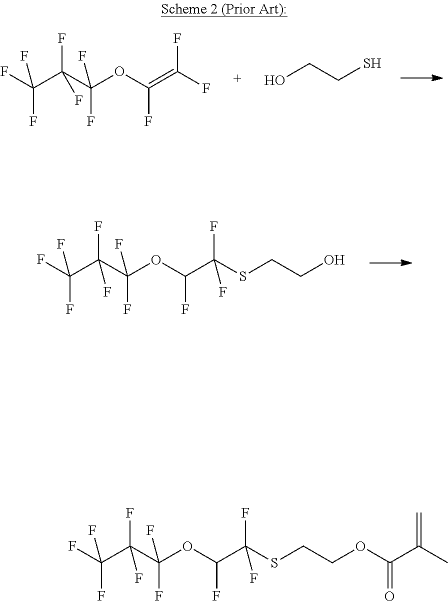

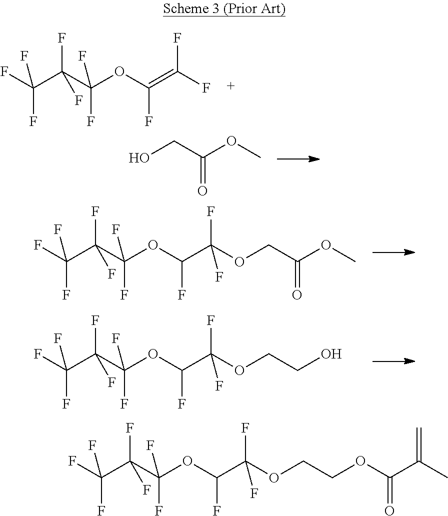

The process according to the present invention can be carried out using reactants and conditions that are known to the person skilled in the art and are described in the literature. The necessary starting materials are commercially available, can be prepared by known processes from commercially available products or can be prepared analogously by known syntheses. Further suitable and preferred reactants and reaction conditions are described in the examples.

The reaction of compounds II and III is preferably carried out in an organic solvent.

Suitable and preferred solvents include linear or cyclic alkyl ethers like dioxane, tetrahydrofurane or methyl-tert-butylether, or mixtures thereof, furthermore aliphatic hydrocarbons or aromatic hydrocarbons like toluene.

The reaction of compounds II and III is carried out in the presence of a base. Suitable and preferred bases include alkali carbonates like Cs₂CO₃, alkaline earth carbonates like CaCO₃, or alkali hydroxides, like KOH or NaOH.

The reaction mixture is preferably heated to a temperature above room temperature, preferably to a temperature of at least 50° C., more preferably at least 80° C., and stirred for a given time, preferably 12 h or more. The heating and stirring are preferably carried out in an inert reaction atmosphere like N₂ or Ar.

The final product can be isolated from the reaction mixture and purified by standard work up procedures that are well known to the skilled person and described in the literature, like distillation, extraction, filtering, washing, drying etc.

Unless stated otherwise, above and below all percentages of solids are percent by weight (“wt. %”), all temperatures are given in degrees Celsius (° C.), “room temperature (RT)” means 20° C., and all physical properties and values refer to a temperature of 20° C.

The invention will now be described in more detail by reference to the following examples, which are illustrative only and do not limit the scope of the invention.

The complete disclosure contents of all applications and publications men-tioned expressly also belong to the disclosure content of the present appli-cation by way of reference. Further features, advantages and variants of the invention also arise from the claims and examples. The following examples explain the present invention in greater detail without restricting the scope of protection.

### EXAMPLE 1

Compound (1) is prepared as follows.

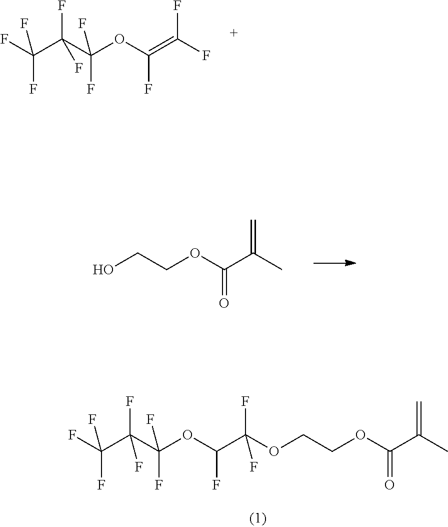

0.255 g (0.78 mmol) of caesium carbonate and 0.01 g (0.05 mmol) of 4-hydroxy-2,2,6,6-tetramethylpiperidin-1-oxyl (TEMPO) are initially introduced in a round-bottomed flask. 0.33 g (2.52 mmol) of 2-hydroxyethyl methacrylate and 0.75 g (2.82 mmol) of perfluoropropylvinyl ether (PPVE) in 3.34 g of dioxane are then added dropwise. The reaction mixture is subsequently heated to 100° C. and stirred at this temperature for 20 h. The product can be obtained directly from the reaction mixture by distillation.

Yield 0.73 g (73%), purity 98%.

### EXAMPLE 2

Compound (2) is prepared as follows.

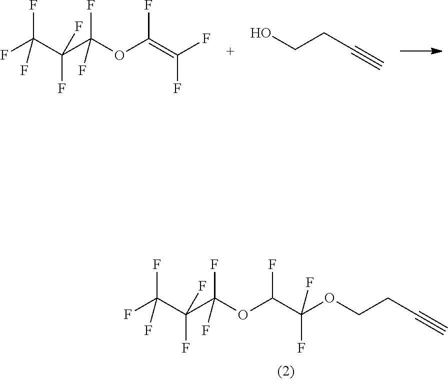

In 150 ml stainless steel pressure reactor 7.96 g (0.03 mol) of Perfluoro propylvinyl ether (PPVE) was added to a solution consisting of 2.52.g (0.04 mol) 3-Butin-1-ol, 2.18 g (0.04 mol) KOH and 25 ml of 1,4 Dioxane. The mixture was heated to 75° C. and kept at this temperature for 20 h. For the work up the reaction mixture was diluted with 30 ml water and 30 ml MTBE, the organic layer was separated and the aqueous layer washed twice with 30 ml MTBE. The combined organic phases were washed with 20 ml saturated NaCl and dried over Na₂SO₄. The product was carefully stripped from the solvents by distilling off the MTBE in a rotary evaporator.

Yield: 4.89 g (48%), purity 95%.

1. A process for preparing a compound of formula I
(Rf(CHF—CF₂Y)ₘ)ₙL(X)ₖ I
by reacting a compound of formula II
Rf(CHF═CF₂)ₘ II
with a compound of formula III
(HY)ₙL(X)ₖ III
in the presence of a base,
wherein the individual radicals, independently of each other and on each occurrence identically or differently, have the following meanings
Rf is a perfluorinated alkyl or alkylene group that optionally contains one or more hetero atoms,
Y is O or S,
L is a single bond or an at least divalent organic group,
X is an ethylenically unsaturated group,
m is 1 or 2,
n is 1, 2 or 3,
o is 1, 2 or 3.

2. The process according to claim 1, characterized in that Rf is a perfluorinated C1-C30 alkyl or alkylene group that optionally contains one or more hetero atoms.

3. The process according to claim 1, characterized in that m is 1 Rf is selected from the following groups
CF₃—(CF₂)₀₋₃—, CF₃—(CF₂)₀₋₃—O—,
CF₃—(CF₂)₀₋₃—O—(CF₂)₁₋₃—,
CF₃—(CF₂)₀₋₃—O—(CF₂)₁₋₃—O—,
CF₃—(CF₂)₀₋₃—O—(CF₂)₁₋₃—O—CF₂—,
CF₃—(CF₂)₀₋₃—O—(CF₂—O)₁₋₈— and
CF₃—(CF₂)₀₋₃—O—(CF₂—O)₁₋₈—CF₂—.

4. The process according to claim 1, characterized in that m is 1 and Rf is selected from the following groups
CF₃—(CF₂)₁₋₂—, CF₃—(CF₂)₁₋₂—O—,
CF₃—O—(CF₂)₁₋₃—,
CF₃—O—(CF₂)₁₋₂—O—,
CF₃—(CF₂)₁₋₂—O—CF₂—,
CF₃—O—(CF₂)₁₋₂—O—CF₂—,
CF₃—O—(CF₂—O)₁₋₈— and
CF₃—O—(CF₂—O)₁₋₈—CF₂—.

5. The process according to claim 1, characterized in m is 2 and Rf is selected from the following groups
—(CF₂)₀₋₄—, —O—(CF₂)₀₋₃—O—,
—(CF₂)₁₋₃—(CF₂)₀₋₃—O—(CF₂)₁₋₃—,
—O—(CF₂)₁₋₃—(CF₂)₀₋₃—O—(CF₂)₁₋₃—O—,
—(CF₂)₁₋₃—O—(CF₂)₁₋₃—O—CF₂—,
—CF₂—(CF₂)₀₋₃—O—(CF₂—O)₁₋₈— and
CF₂—(CF₂)₀₋₃—O—(CF₂—O)₁₋₈—CF₂—.

6. The process according to claim 1, characterized in that m is 2 and Rf is selected from the group consisting of
—(CF₂)₁₋₃—, —(CF₂)₁₋₃—O—,
—O—(CF₂)₁₋₃—O—,
—CF₂—O—(CF₂)₁₋₂—O—,
—CF₂—(CF₂)₁₋₂—O—CF₂—,
—CF₂—O—(CF₂)₁₋₂—O—CF₂—,
—CF₂—O—(CF₂—O)₁₋₈— and
—CF₂—O—(CF₂—O)₁₋₈—CF₂—.

7. The process according to claim 1, characterized in that X is an acrylate or methacrylate group or an ethynyl group.

8. The process according to claim 1, characterized in that L is a single bond or a saturated C1-C20 alkylene group that is straight-chain or branched, optionally contains one or more heteroatoms, preferably one or more O atoms, and optionally contains one or more functional groups.

9. The process according to claim 1, characterized in that L is selected from the group consisting of methylene, ethylene, propylene, isopropylene, n-butylene, isobutylene, sec-butylene, t-butylene, or pentylene or hexylene which are straight-chain or branched.

10. The process according to claim 1, characterized in that
m is 1 and Rf is selected from CF₃—(CF₂)₁₋₂—, CF₃—(CF₂)₁₋₂—O—, CF₃—O—(CF₂)₁₋₃—, CF₃—O—(CF₂)₁₋₃—O—, CF₃—(CF₂)₁₋₂—O—CF₂—, CF₃—O—(CF₂)₁₋₂—O—CF₂—, CF₃—O—(CF₂—O)₁₋₈— and CF₃—O—(CF₂—O)₁₋₈—CF₂—,
or m is 2 and Rf is selected from —(CF₂)₁₋₄—, —O—(CF₂)₁₋₃—O—, —(CF₂)₁₋₃—(CF₂)₀₋₃—O—(CF₂)₁₋₃—, —O—(CF₂)₁₋₃—(CF₂)₀₋₃—O—(CF₂)₁₋₃—O—, —(CF₂)₁₋₃—O—(CF₂)₁₋₃—O—CF₂—, —CF₂—(CF₂)₀₋₃—O—(CF₂—O)₁₋₈— and CF₂—(CF₂)₀₋₃—O—(CF₂—O)₁₋₈—CF₂—, and
L is a C1-C6 alkylene group that is straight-chain or branched and optionally contains one or more hetero atoms, preferably one or more O atoms, and optionally contains a functional group.

11. The process according to claim 1, characterized in that the compounds of formula I are selected from the following formulae: 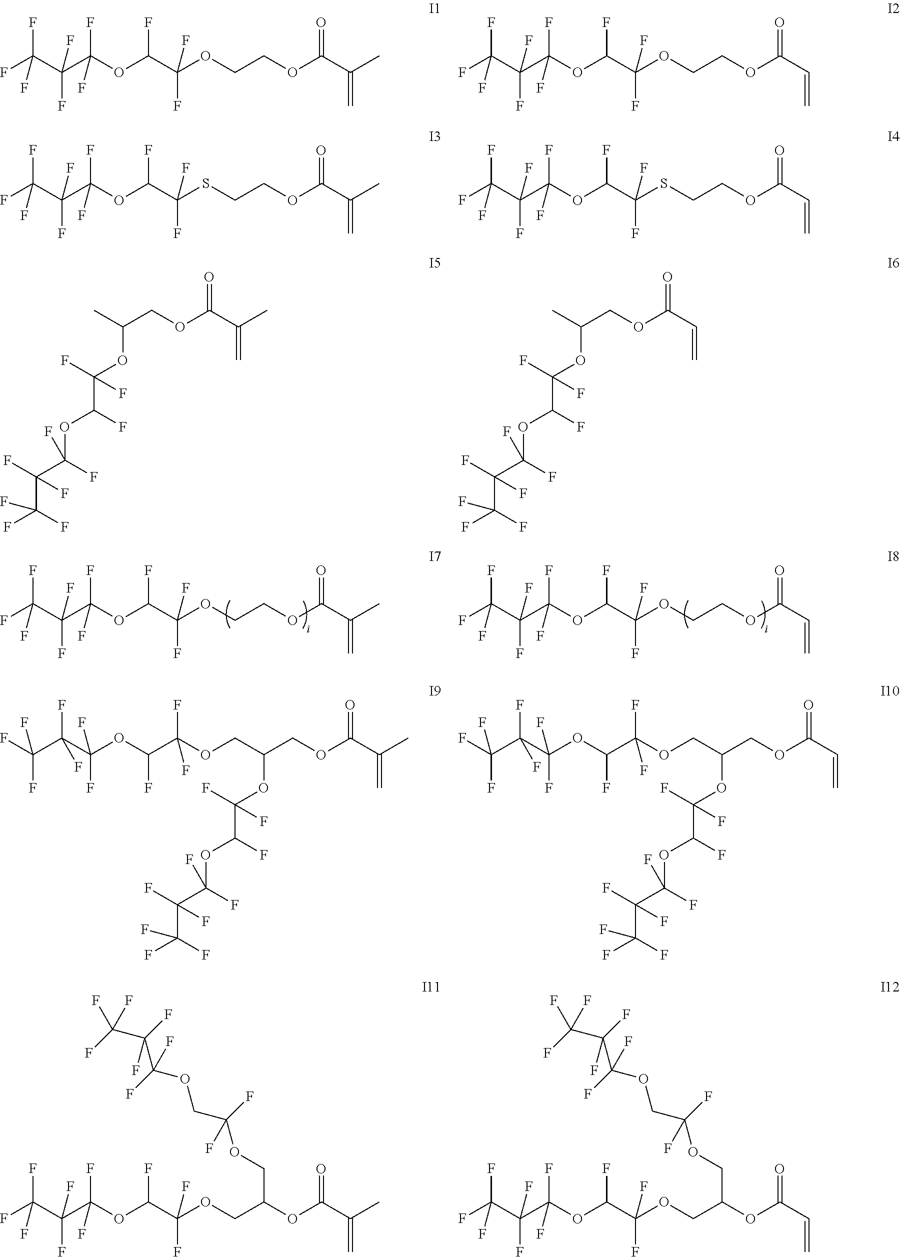 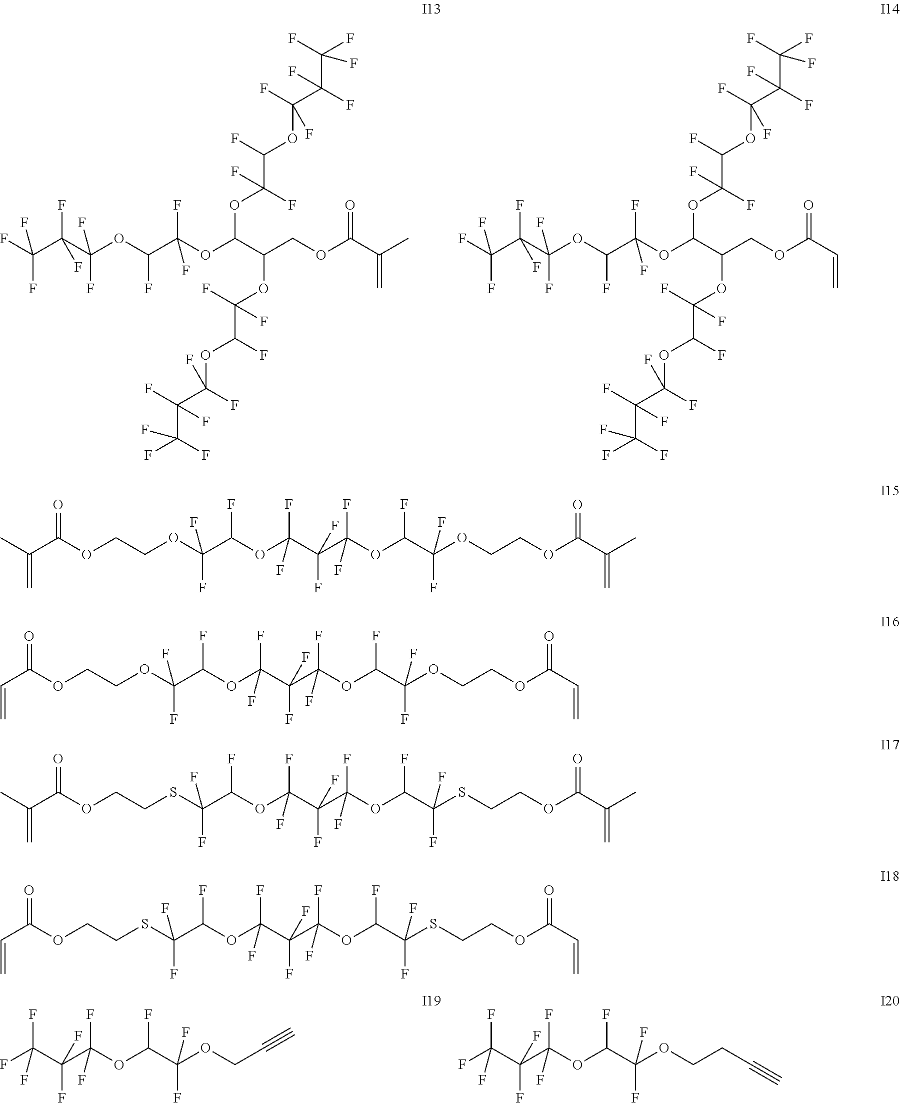
wherein i is 2-10.

12. The process according to claim 1, characterized in that the compounds of formula II are selected from the following formulae:

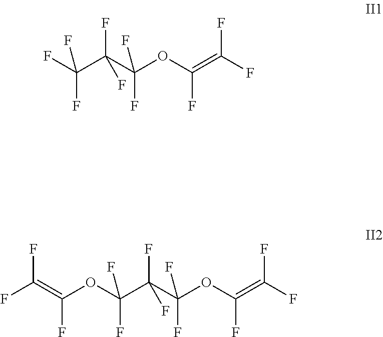 [SMILES: FC(F)=C(F)OC(F)(F)C(F)(F)C(F)(F)F ; FC(F)=C(F)OC(F)(F)C(F)(F)C(F)(F)OC(F)=C(F)F]

13. The process according to claim 1, characterized in that the compounds of formula III are selected from the following formulae: 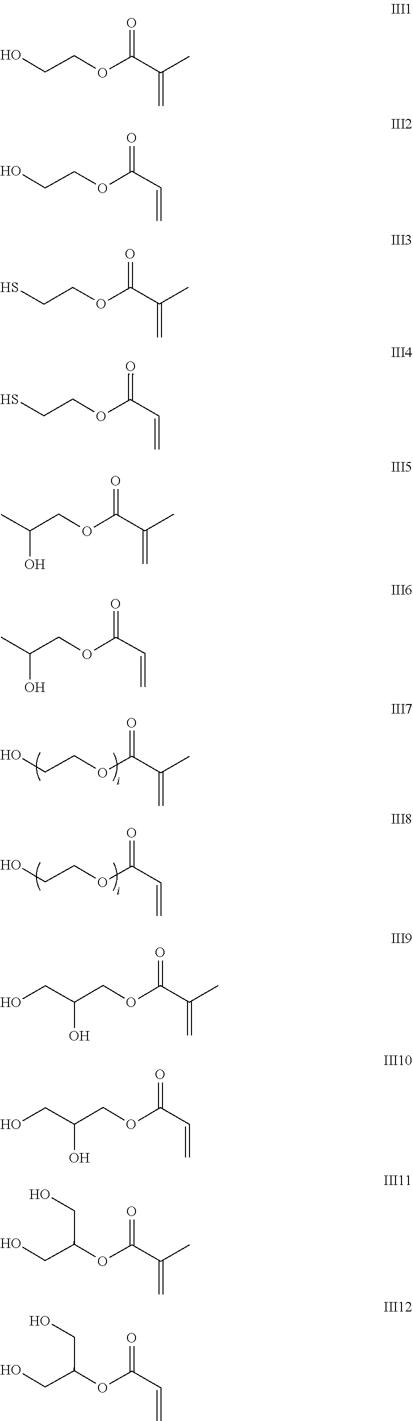 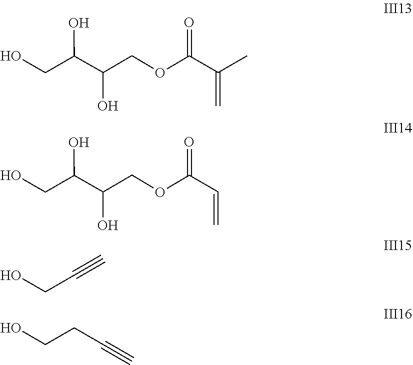
wherein i is 2-10.

14. The process according to claim 1, characterized in that the reaction of compounds II and III is carried out in an organic solvent selected from linear or cyclic alkyl ethers, or aliphatic or aromatic hydrocarbons.

15. The process according to claim 14, characterized in that the solvent is selected from dioxane, tetrahydrofurane or methyl-tert-butylether, or mixtures thereof, or toluene.

16. The process according to claim 1, characterized in that the base is selected from alkali carbonates, alkaline earth carbonates or alkali hydroxides.

17. The process according to claim 1, characterized in that the reaction mixture is heated to a temperature above room temperature and stirred.

18. A compound of formula I or its subformulae as defined in claim 1, wherein X is an ethynyl group.
